(19) United States
(12) Patent Application Publication
(10) Pub. No.: US 2013/0053753 A1
(43) Pub. Date: Feb. 28, 2013

(54) CATHETER TIP

(71) Applicants: Eric M. King, West Jordan, UT (US); Ron Wortley, Salt Lake City, UT (US)

(72) Inventors: Eric M. King, West Jordan, UT (US); Ron Wortley, Salt Lake City, UT (US)

(21) Appl. No.: 13/601,321

(22) Filed: Aug. 31, 2012

Related U.S. Application Data
(60) Provisional application No. 61/529,626, filed on Aug. 31, 2011.

(51) Int. Cl. A61M 25/14

(52) U.S. Cl. 604/6.16

(57) ABSTRACT

Catheters and their uses for vascular access to a patient are described. The catheter comprises an arterial lumen and a venous lumen which run along the shaft of the catheter for most of the length. Near the catheter tip, the two lumens can twist around the longitudinal axis of the catheter. The catheter shaft has two openings, one where the arterial lumen begins the twist and another about 180 degrees into the twist. The catheter shaft also contains an opening for the venous lumen near the nose of the catheter shaft. The orientation of the arterial lumen opening on opposite sides of the shaft eliminates the possibility of side port occlusions since the catheter can't be pressed against a vessel wall that blocks both suction openings simultaneously. And using these openings at each point reduces the chance of occlusion due to blood clots and fibrin sheaths. Other embodiments are described.

### CROSS-REFERENCE TO RELATED APPLICATION

This patent application claim priority of U.S. Provisional Application Ser. No. 61/529,626, filed on Aug. 31, 2011, the entire disclosure of which is hereby incorporated by reference.

### FIELD

This application generally relates to medical devices and methods for making and using such medical devices. More particularly, this application relates to medical devices used for vascular access, commonly known as a catheter, and methods for making and using such medical devices for vascular access to a patient.

### BACKGROUND

Hemodialysis is a treatment designed to clean the blood of waste and excess fluid when a patient suffers from impaired kidney function. Proper access to the patient's blood and transport of the blood to and from the dialysis machine can be important during hemodialysis. Access to the blood can gained through the use of a dialysis catheter that is placed inside the patient's vascular system, typically near the heart.

Most dialysis catheters contain three main components including the catheter shaft, the hub (or bifurcation), and extension tube(s). The catheter shaft is that portion of the catheter which is inserted into the vascular system and provides subcutaneous access. The catheter shaft can sometimes contain a single lumen that forms a single flow path. Alternatively, the catheter shaft can contain multiple lumens that form several flow paths. These multiple lumens are often categorized as the arterial (or inlet) lumen(s) and the venous (or outlet) lumen(s). The arterial inlet lumen(s) transports blood from the patient to the dialysis machine and uses negative pressure to suck or pump blood out of the body and into the machine. In contrast, the venous outlet lumen(s) transports blood back from the machine to the patient and operates using a positive pressure suck or pump blood out of the machine and into the body.

### SUMMARY

The application describes catheters, methods for making them, and methods for using them to provide vascular access to the body of a patient. The catheter contains a catheter shaft with two lumens, one for removing blood from the body and one for returning blood to the body. The two lumens can run substantially parallel to each other as they extend along the shaft of the catheter for the majority of its length. Near the tip of the catheter, the two lumens can twist or rotate around the linear axis of the catheter. The first (or arterial) lumen has two openings, one near the point where the lumen begins to twist and another opening on an opposite side of the lumen. The venous lumen contains an opening near the distal end (or nose) of the catheter shaft. The orientation of the openings of the arterial lumen on opposite sides of the shaft reduces or eliminates the possibility of inlet flow restrictions or occlusions since at no point can the catheter be positioned against a vessel wall or other part of the vascular anatomy that would block both inlet openings at the same time. Additionally, using full-sized openings at each point reduces the chance of occlusion due to blood clots and/or fibrin sheaths.

### BRIEF DESCRIPTION OF THE DRAWINGS

The following description can be understood in light of FIGS. 1-24, in which:

FIG. 1 illustrates a view of some embodiments of a multi-lumen catheter;

FIG. 2 illustrates some embodiments of the distal end portion of a multi-lumen catheter;

FIG. 3 illustrates a cross sectional view of some embodiments of a multi-lumen catheter;

FIGS. 4-7 illustrate a cross sectional view showing some embodiments of a multi-lumen catheter;

FIGS. 8-10 illustrate various views of some embodiments of the distal end portion of a multi-lumen catheter that contains arterial intake openings oriented on opposite sides of the catheter shaft;

FIGS. 11-13 illustrate several views of some embodiments of the distal end portion of a lumen in a multi-lumen catheter;

FIGS. 14-17 illustrate several views of some embodiments of the distal end portion of a multi-lumen catheter in which the lumens twist into a helix;

FIGS. 18-22 illustrate cross sectional views taken along their respective lines of FIG. 15;

FIG. 23 illustrates a cross sectional view taken along line 23 of FIG. 9; and

FIG. 24 illustrates a cross sectional view taken along line 24 of FIG. 10.

The Figures illustrate specific aspects of the catheters, methods for making them, and methods for using them to provide vascular access to the body of a patient. Together with the following description, the Figures demonstrate and explain the principles of the methods and structures produced through these methods. In the drawings, the thickness of layers and regions are exaggerated for clarity. The same reference numerals in different drawings represent the same element, and thus their descriptions will not be repeated. As the terms on, attached to, or coupled to are used herein, one object (e.g., a material, a layer, a substrate, etc.) can be on, attached to, or coupled to another object regardless of whether the one object is directly on, attached, or coupled to the other object or there are one or more intervening objects between the one object and the other object. Also, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements.

### DETAILED DESCRIPTION

The following description supplies specific details in order to provide a thorough understanding. Nevertheless, the skilled artisan would understand that the catheter devices and associated methods of making and using the devices can be implemented and used without employing these specific details. Indeed, the catheter devices and associated methods can be placed into practice by modifying the illustrated devices and methods and can be used in conjunction with any other apparatus and techniques conventionally used in the industry. For example, while the description focuses on a bi-lumen catheter, it could easily be adapted to be used with any number of lumens. As well, while the description focuses on a dialysis catheter, it could be modified and used for any medical procedure or device that requires or functions under a negative flow, including drainage catheters, PICC catheters, and Cardiac Bypass catheters.

Some embodiments of the catheters, methods for making them, and methods for using them to provide vascular access to the body of a patient. In these embodiments, a catheter can be provided that includes a catheter shaft, a hub (or bifurcation), and extension tubes which can be connected to a dialysis machine. The catheter shaft can be divided into two or more lumens, providing for one or more directions of flow (to and/or from) the patient's vascular system. The lumen(s) can run the full length of the catheter shaft and then terminate with an opening substantially near the distal end of the catheter.

The catheter shaft and the lumens can be made of any material. In some configurations, the catheter shaft can contain barium sulfate, titanium dioxide, tungsten, and/or other radio-opaque substances. These substances can help define the location of the catheter shaft in the vascular system when it is subjected to fluoroscopy, X-rays, or other medical imaging.

The catheter can be used to provide access to the vascular system. In some configurations, the catheter shaft can be tunneled under the skin and then maneuvered into the desired location of the patient's body (i.e., the desired vein or other part of the vascular anatomy). Prior to inserting the catheter shaft into the body, in some configurations a guide wire can be introduced that will help steer the catheter shaft into this desired position. The catheter shaft can be inserted along the guide wire and guided into place using the fluoroscopy, X-rays, or other medical imaging.

In some embodiments, the arterial (or intake) lumen(s) operate under a negative pressure. In these embodiments, the arterial lumen(s) transports blood from the individual to a dialysis machine using this negative pressure, thereby removing or pumping blood out of the body and into the machine via the arterial inlet opening in the catheter shaft. In these embodiments, the venous outlet (or return) lumen(s) operates under a positive pressure and can be used to transport that blood back from the dialyses machine to the patient via the venous return opening in the catheter shaft. In other embodiments, the arterial and the venous lumens can be reversed by switching the arterial and venous connections on the dialysis machine. The configuration is these embodiments can be used to try and flush fibrin from the arterial line/tip.

In some embodiments, the catheter shaft can be configured to create two lumens that run substantially parallel to each other along the length of the catheter shaft. In other embodiments, the catheter shaft can be divided into three, four, or even more lumens that extend substantially parallel to each other along the length of the catheter shaft. The lumens can extend along part or all of the length of the catheter shaft.

In other configurations, the catheter shaft can be divided into two or more lumens so that a portion of the lumen(s) does not extend not parallel to the catheter shaft, especially near the tip. For example, the catheter shaft can be divided into two lumens where a portion of these two lumens twist in a helix about the longitudinal axis of the catheter shaft near the tip. In these configurations, the two lumens can twist, weave, angle, split, or otherwise oriented in various directions away from and/or toward the longitudinal axis of the catheter shaft. Thus, the catheter shaft can contain one or more sections in which the lumen(s) deviates from a course parallel to the longitudinal axis of the catheter shaft.

In some configurations, the lumen(s) in the catheter shaft can terminate with one or more openings near the distal end (i.e., the nose) of the catheter. The arterial inlet opening(s) and/or the venous return opening(s) in the lumen(s) can be placed at any desired location along the catheter shaft. In some configurations, the venous return opening(s) can be located near the catheter's nose and the arterial intake opening(s) can be located any distance farther up the catheter shaft. In other configurations, the openings they can be located oppositely, i.e. the opening can be located in the tip of arterial lumen and along the shaft of the venous lumen. These other configurations could adversely affect the recirculation since a percentage of the blood exiting the arterial lumen could be drawn right back into the venous lumen, resulting in decreased dialysis efficiency.

In some configurations, the lumen(s) may be configured to have one or more openings oriented at any distance along the catheter shaft. In some embodiments, the one or more openings can be located at about 5 to about 15 m from the distal end of the catheter shaft. In other embodiments, the one or more openings can be located at about 20 to about 30 mm from the distal end of the catheter shaft. In yet other embodiments, the one or more openings can be located at any combination or sub-range of these amounts from the distal end of the catheter shaft.

In some configurations, the catheter shaft can have multiple openings oriented on various positions or sides of the lumens. For example, the catheter can be configured to that there are two openings located on nearly opposite sides of the catheter shaft. In another example, the catheter can contain three (or more) openings where only two of the openings are located on nearly opposite sides of the catheter shaft.

In some embodiments, the lumen(s) may have one or more openings oriented along a line substantially parallel to the longitudinal axis of the catheter shaft (i.e., at about the same location around the circumference of the shaft). In other embodiments, the lumen(s) may have one or more openings oriented in a pattern such that the openings are situated between about 1 and about 359 degrees apart from each other, as measured from the longitudinal axis of the catheter shaft. In yet other embodiments, the lumen(s) may have one or more openings oriented in a pattern such that the openings are situated between 5 and 45 degrees apart from each other, as measured from the longitudinal axis of the catheter shaft. In other embodiments, the lumen(s) may have one or more openings oriented in a pattern such that the openings are situated about 180 degrees apart from each other. In still other embodiments, the one or more openings can be oriented at any combination or sub-range of these amounts.

The lumen(s) can contain as many openings as desired that allows the lumen(s) to operate as described herein. In some embodiments, the shaft can contain 2 (or more) openings for the arterial lumen. In other embodiments, the shaft can contain 1 opening for the venous lumen. In yet other embodiments, these could be reversed so that the venous contains two or more openings and the arterial lumen just contains one.

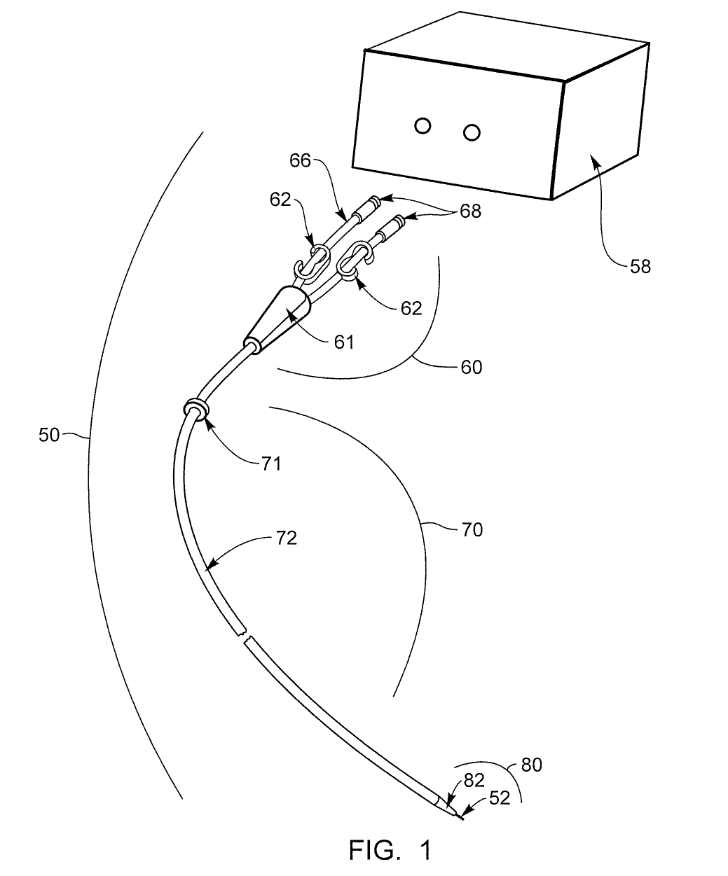

Some embodiments of multi-lumen catheters are illustrated in FIG. 1. FIG. 1 depicts a catheter 50 comprised of a proximal end portion 60, a middle (or intermediate) portion 70, and a distal end portion 80. The proximal end portion 60 of the catheter 50 contains a hub 61 that connects the extension tubes (i.e., arterial intake extension tube 64 and the venous return extension tube 66) to the catheter shaft 72. These extension tubes 64 and 66 can contain threads 68 (or other connection mechanism) that are used to connect them to the dialysis machine 58. In some configurations, a clamping mechanism 62 can be placed on the desired location of the extension tubes and used to stop and start the blood flow within the extension tubes 64 and 66.

In the middle portion 70 of the catheter 50, the catheter shaft 72 can be connected to the hub 60. The catheter shaft 72 extends down to the distal end portion 70 of the catheter 50. In some configurations, a fibrous cuff 71 (or other suture mechanism) can be positioned a short distance away from the hub 60 to aid in closing the site at which the catheter 50 enters the patient's body. The shaft can be any shape consistent with this function, including substantially your or oval. In some configurations, the shaft could be made of polyurethane or silicone.

At the distal end portion 80 of the catheter 50, the catheter shaft 72 can end in a nose section 82. The nose section is the first part of the catheter 50 that is inserted in the patient and can be configured with this purposed in mind. Thus, in some configurations, the catheter shaft 52 can contain a guide wire hole 53 in the nose 82 that allows a guide wire 52 to be threaded into the catheter shaft 72 and thus used to assist placing the catheter in the correct position of the patient. In other configurations, the nose section can be configured to cooperate with a tunneling system that can be used to place the catheter in the desired location. To work with the tunneling system, the physician typically makes a first incision in the catheter entry point, i.e., the access point in the skin and in the vessel wall. A second incision is also made at the catheter exit point in the skin a short distance away from the first incision. The nose section 82 of the catheter 50 is then attached to a tunneler which is inserted into the second incision and pushed under the skin until it exits the first incision, thereby creating a tunnel between the two incisions. The tunneler is then used to pull the catheter through the tunnel underneath the skin. The nose section 82 is then removed from the tunneler and the catheter is pushed back through the vascular access incision into the vessel incision, thereby allowing external access to the vascular system.

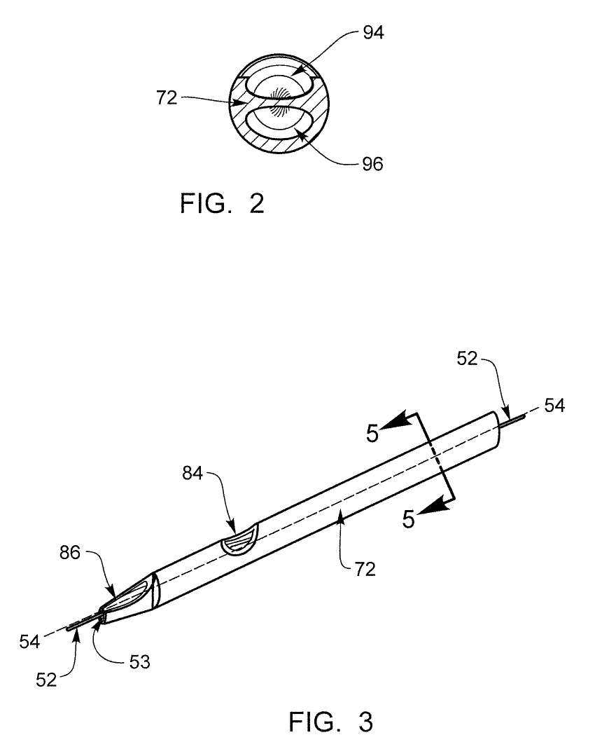

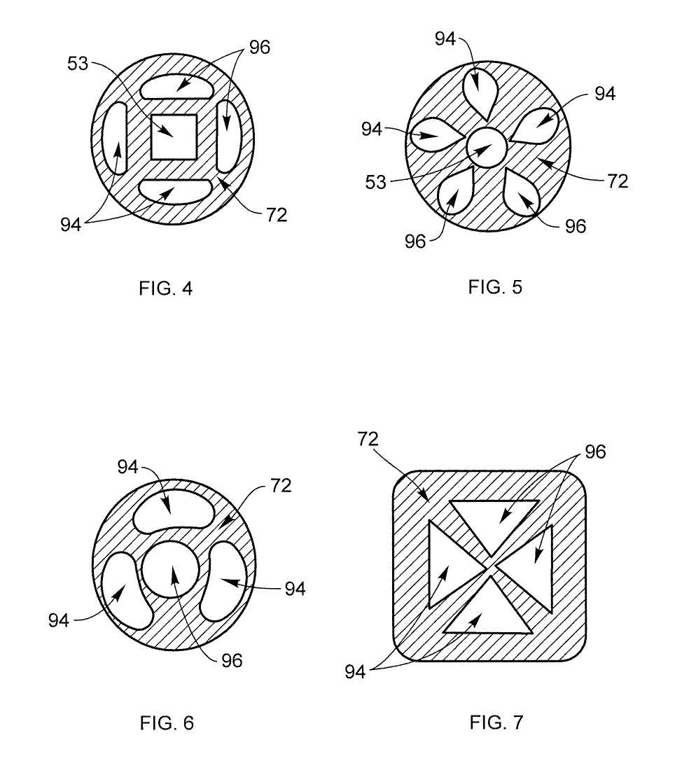

FIGS. 2 and 3 illustrate a close-up view of some configurations of the catheter shaft 72. In these configurations, the catheter shaft 72 can contain two lumens, such as an arterial intake lumen 94 and a venous return lumen 96. The lumens can have any shape that allows blood to flow through it. FIG. 4 is a cross-section view (transverse to the longitudinal axis 54 of the catheter shaft 52) that illustrates some configurations where one and/or both of the lumens inside of the catheter shaft 72 can be substantially D-shaped. FIGS. 5, 6, and 7 illustrate other embodiments where one and/or both of the lumens inside the catheter shaft 72 can have other cross-sectional shapes. As shown in these four figures, the lumens can have substantially similar shapes (as shown in FIGS. 4, 5, and 7) or can have different shapes from each other (as shown in FIG. 6).

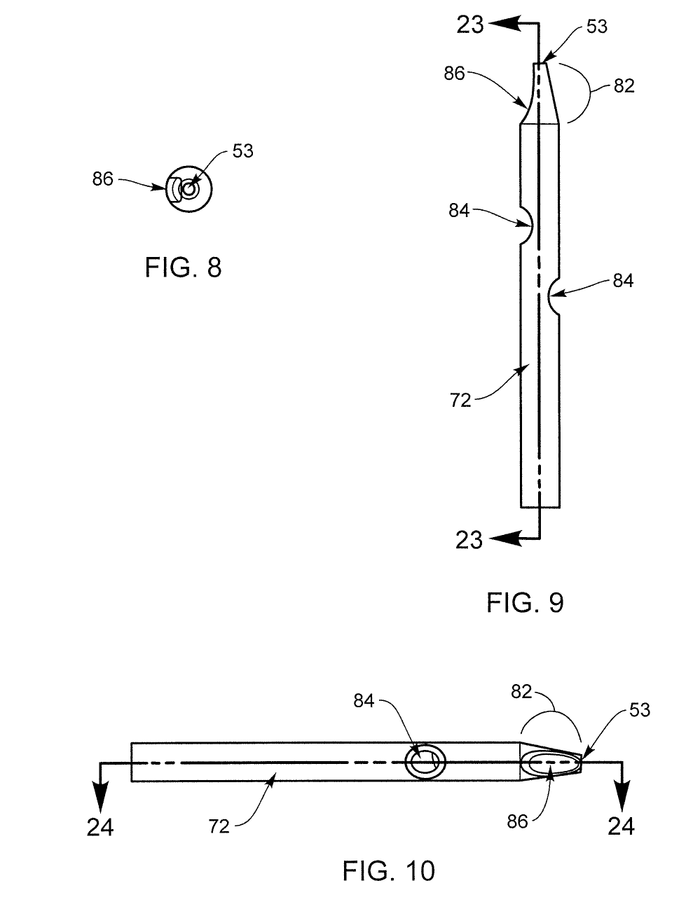

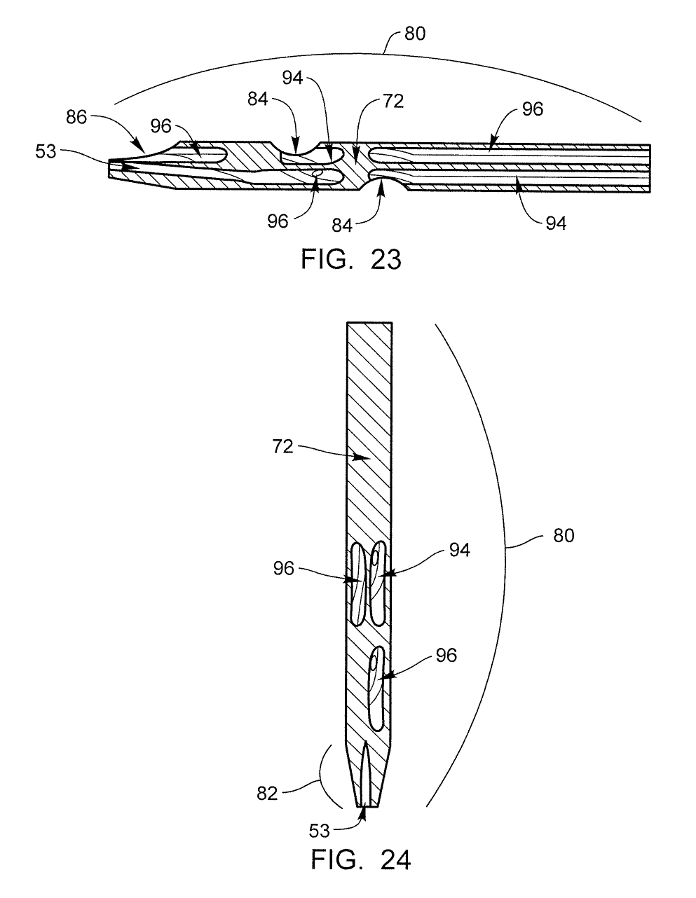

One or more openings can be located along one or both of the catheter lumens and/or in one or both of the nose section. For example, as illustrated in FIG. 3, one or more arterial intake openings 84 can be located along length of the arterial lumen 94 and/or one or more venous return openings 86 can located near the end of the venous lumen 96. FIGS. 8, 9, and 10 (and their cross-sections shown in FIGS. 23-24) illustrate those configurations where the catheter lumen(s) contain multiple openings. As shown in these figures, the catheter shaft 72 can be configured to contain two arterial intake openings 84 that are situated substantially 180 degrees apart as measured around the longitudinal axis 54 (i.e., they are located on nearly opposite sides around the circumference of the catheter shaft 92). The catheter shaft 72 can also be configured to contain a venous return opening 86 that is located near the end of the venous lumen (i.e., the section nose 82).

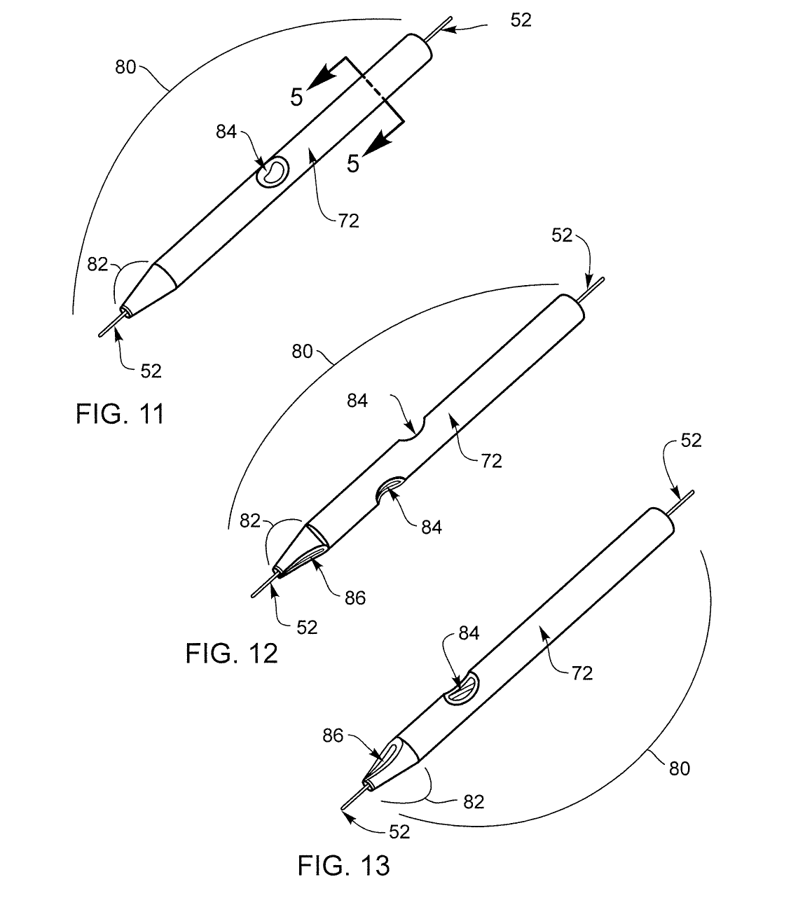

FIGS. 11, 12, and 13 illustrate several views of a catheter shaft in the embodiments containing multiple openings as it rotates about its axis. As shown in FIGS. 11, 12, and 13, the catheter shaft 72 can contain a first opening 84 on one side of the shaft 72, as well as a second opening 84 and a third opening 84 on an opposite side of the shaft 72. The second opening 84 is located closer to the nose section 82 than the first opening and both the second and third openings open into the arterial lumen. The third opening is located in the nose section of the catheter shaft 72 and opens into the venous lumen.

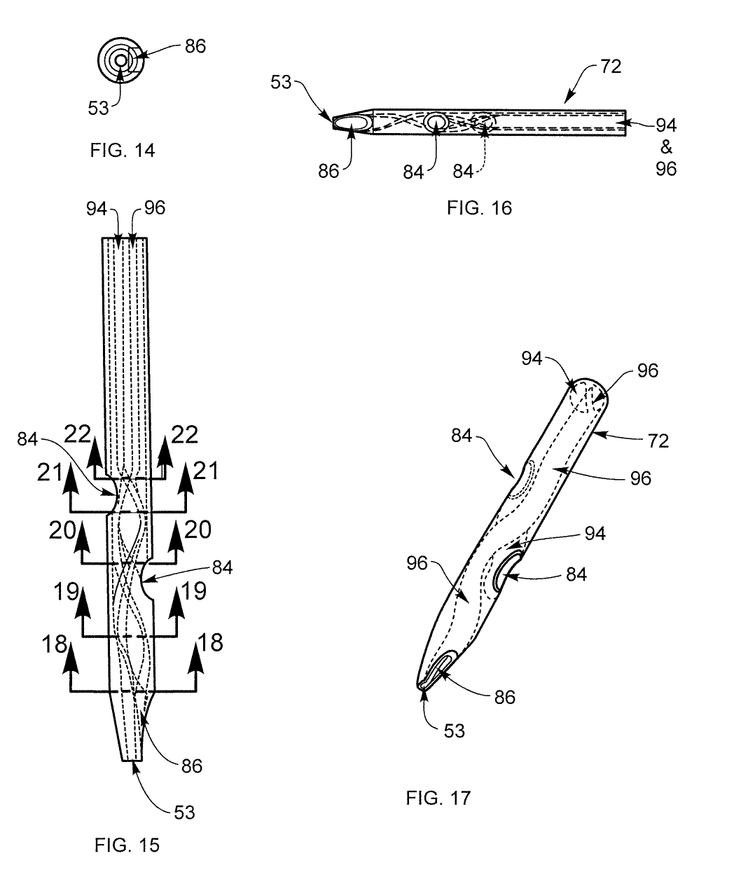

In some configurations, the lumens can extend within the catheter shaft 72 parallel to the longitudinal axis 54 of the catheter shaft. In other configurations, though, the lumens do not travel in a path within the catheter shaft 72 that remains parallel to the longitudinal axis 54, as illustrated by FIGS. 14-17. In these other configurations, the arterial intake lumen 94 and/or the venous return lumen 96 can twist about the longitudinal axis 54. In some embodiments, the arterial intake lumen 94 and/or the venous return lumen 96 can twist in a helix about the longitudinal axis 54.

The lumen(s) can twist around longitudinal axis 54 any desired amount. In some configurations, the lumen(s) can twist around longitudinal axis 54 up to about 360 degrees, up to about 270 degrees, up to about 180 degrees, and/or even about up to 90 degrees. In other embodiments, the twist can twist more than about 360 degrees, more about 720 degrees, and/or can include multiple twists of varying degrees. In yet other embodiments, the lumen(s) can twist any combination or sub-range of these amounts.

In the configurations where a lumen contains multiple openings, it can be configured to twist so that one or more of the openings are exposed. In some embodiments, the arterial intake lumen 94 can be twisted so that the two or more arterial intake openings 84 along the length of the shaft are exposed with one of the intake openings 84 located closer to the nose section 82 than the other opening. And the venous return lumen 96 can be twisted so that it terminates in a venous return opening 86 near the nose section 82.

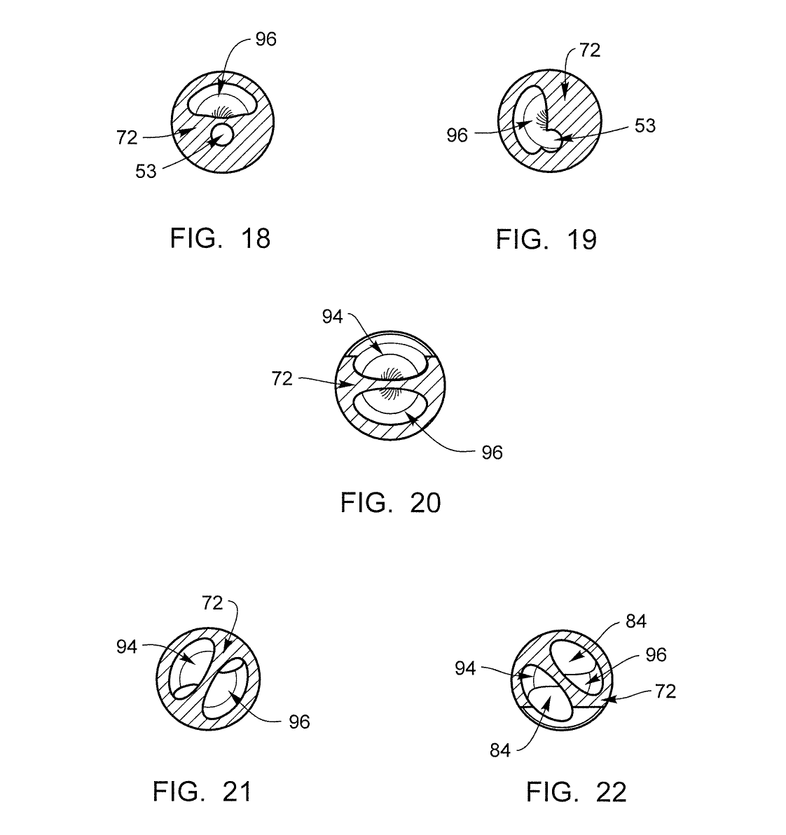

In some configurations, the arterial intake lumen(s) 94 and/or the venous return lumen(s) 96 rotate about the longitudinal axis 54 of the catheter shaft 72 so that the position of the lumens 94 and/or 96 relative to the longitudinal axis 54 can rotate anywhere between 0 and about 360 degrees. For example, as shown by the transverse cross sections of various embodiments shown in FIGS. 18, 19, 20, 21, and 22 (which are cross-sections taken along the length of FIG. 15), the arterial intake lumen 94 rotates about the longitudinal axis 54 of the catheter shaft 72 so that the position of the arterial intake lumen 94 relative to the longitudinal axis 54 rotates about 180 degrees, and the venous return lumen rotates 360 degrees. In such configurations, the arterial intake lumen 94 is exposed in two separate arterial intake openings 84 that are nearly 180 degrees apart on nearly opposite sides of the catheter shaft 72 (with one arterial intake opening closer to the nose 82) and the venous return lumen 96 is exposed in a venous return opening 86 near the nose section 82.

With multiple openings in a lumen(s) and with a twist, these catheters can be much safer in terms of the risk of occlusion and low efficiency. When foreign materials are introduced into the body of a patient, the body often attempts to reject the foreign material by growing fibrin around it. But large arterial intake openings and venous return openings are less easily blocked by such fibrin growth. And by placing large arterial intake openings on both sides of the catheter shaft, the lumen(s) is less likely to be blocked by fibrin growth, blood clots, or other materials, thereby increasing the efficiency of transporting blood to and from the patient.

Different sizes and/or shapes of the opening can be used. In some configurations, the sizes and/or shapes can be changed to match the desired pressure drop over the length of the catheter to balance the inlet flow. In other configurations, the sizes and/or shapes of the openings can be changed to prevent complete occlusion.

In some conventional catheters, the arterial lumen functions under a negative flow pressure. Thus, there exists a significant risk of an arterial flow restriction or complete obstruction, resulting in loss of flow because the arterial inlet comes in contact with (or sucking up against) a vessel wall or some other part of the patient's vascular anatomy. For this reason, side holes in some conventional catheters are often offset along the length of the catheter shaft to help reduce the occurrence of the arterial flow restrictions or occlusions. These side holes function as secondary inlets to redirect negative pressure flow if the primary arterial inlet becomes obstructed or occluded. But these offset side holes in the conventional catheters are only positioned on the arterial side of the catheter shaft. As such, the risk of these offset side holes also becoming obstructed or occluded by coming in contact with a vessel wall or some other part of the patient's vascular anatomy, while reduced, remains significant and a major concern for the efficiency and function.

Moreover, the arterial intake lumen use negative pressure to draw blood from the body to the dialysis machine. So if the arterial intake opening becomes blocked by an object (such as a vein wall), the flow of blood may be reduced or even stopped complete, placing the patient at risk. By locating the arterial intake openings on different sides (even if not opposite sides) of the catheter shaft, the negative pressure inside the arterial intake lumen cannot create enough suction to completely occlude both openings.

In some configurations, the twist in the catheter lumens can provide other benefits. Where the lumen(s) are twisted about 360 degrees, it can create a flow path geometry that, at least for a standard dialysis catheter, can allow for an arterial side opening to exist on the venous side of the catheter (or can allow a venous side opening to exist on the arterial side of the catheter). These configurations allow two or more arterial side holes or two or more venous side holes to exist on both sides of the catheter which, in some configurations, can be located substantially 180 degrees apart from each other. Thus, in terms of positive and negative flow, the arterial and venous lumens can switched from one side of the catheter to the other side of the catheter for a specific length or mutable lengths of the catheter.

These catheters could be manufactured using any method that provides the structures described herein. In some embodiments, the catheters could be manufactured by extruding the catheter shaft tube to form the twist(s) in the lumens by means of a rotating cross head or other post extrusion/procure operation while the extrusion is exiting the die and/or head. The twist(s) could also be formed with a secondary thermal process such as an RF die or some other heat source in which the twist is formed by inserting corresponding twisted lumen mandrels into the extrusion lumens while applying heat causing the extrusion lumens to take the shape (twist) of the mandrels.

The catheters described above could be modified in various ways. For example, it could easily be adapted for any number of openings, with greater or lesser degree of twist with more or fewer openings along the lumen. For example, the lumens could be twisted in degrees more than 360 degrees so that numerous openings (such as 3 or 4) are separated from each other along the circumference of the shaft. As well, the openings can be located along any desired part of the length of the shaft.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, examples are meant to be illustrative only and should not be construed to be limiting in any manner.

1. A catheter shaft, comprising:
an arterial lumen; and
a venous lumen;
wherein the catheter shaft comprises multiple openings and the arterial lumen is twisted about a longitudinal axis of the catheter shaft so that it is exposed at two different locations around the circumference of the shaft.

2. The shaft of claim 1, wherein a portion of the arterial lumen twists helically about a longitudinal axis of the catheter shaft.

3. The shaft of claim 1, further comprising an opening near the end of the catheter shaft for the venous lumen.

4. The shaft of claim 1, wherein the twist of the arterial lumen can range up to about 360 degrees.

5. The shaft of claim 4, wherein the twist of the arterial lumen can exist at various locations along the length of the catheter shaft.

6. The shaft of claim 1, wherein the venous lumen also twists about a longitudinal axis of the catheter shaft.

7. The shaft of claim 1, wherein the two openings expose the arterial lumen at nearly opposite locations around the circumference of the shaft.

8. The shaft of claim 7, wherein the two openings are not occluded at the same time by the anatomy of the patient.

9. The shaft of claim 1, wherein the arterial lumen is twisted more than 360 degrees.

10. A catheter, comprising:
an arterial extension tube;
a venous extension tube; and
a catheter shaft comprising an arterial lumen and a venous lumen, wherein the catheter shaft comprises multiple openings and the arterial lumen is twisted about a longitudinal axis of the catheter shaft so that it is exposed at two different locations around the circumference of the shaft.

11. The catheter of claim 10, wherein a portion of the arterial lumen twists helically about a longitudinal axis of the catheter shaft.

12. The catheter of claim 10, further comprising an opening near the end of the catheter shaft for the venous lumen.

13. The catheter of claim 10, wherein the twist of the arterial lumen can range up to about 360 degrees.

14. The catheter of claim 13, wherein the twist of the arterial lumen can exist at various locations along the length of the catheter shaft.

15. The catheter of claim 10, wherein the venous lumen also twists about a longitudinal axis of the catheter shaft.

16. The catheter of claim 10, wherein the two openings expose the arterial lumen at nearly opposite locations around the circumference of the shaft.

17. The catheter of claim 16, wherein the two openings are configured to not be occluded at the same time by the anatomy of the patient.

18. The catheter of claim 10, wherein the arterial lumen is twisted more than about 360 degrees.

19. The catheter of claim 10, wherein the shaft comprises two openings that expose the arterial lumen at two locations that are not the same around the circumference of the shaft.

20. The catheter of claim 10, wherein the twist in the arterial lumen places an arterial side hole on the venous side of the catheter,

21. The catheter of claim 15, wherein the twist in the venous lumen places a venous side hole on the arterial side of the catheter.

22. The catheter of claim 10, wherein the arterial lumen is twisted up to about 180 degrees.

23. The catheter of claim 10, wherein the arterial lumen is twisted up to about 90 degrees.
